(19) United States
(12) Patent Application Publication
(10) Pub. No.: US 2025/0305953 A1
(43) Pub. Date: Oct. 2, 2025

(54) LIGHT-EMITTING RADICAL CATION PROBE IN SITU GENERATED BY ACID INDUCTION, AND PREPARATION THEREFOR AND APPLICATION THEREOF

(71) Applicant: SOUTH CHINA UNIVERSITY OF TECHNOLOGY, Guangzhou City (CN)

(72) Inventors: Ben Zhong TANG, Guangzhou City (CN); Meng GAO, Guangzhou City (CN); Shuhui DAI, Guangzhou City (CN)

(21) Appl. No.: 18/563,170

(22) PCT Filed: Apr. 25, 2023
(86) PCT No.: PCT/CN2023/090638

§ 371 Date: Nov. 21, 2023

(30) Foreign Application Priority Data
Dec. 21, 2022 (CN) 202211660940.4

(51) Int. Cl. G01N 21/64; A61K 49/00; C07D 207/333; C07D 211/10

(52) U.S. Cl. CPC G01N 21/6428; A61K 49/0021; C07D 207/333; C07D 211/10; G01N 21/6456; G01N 2021/6439

(57) ABSTRACT

The present invention belongs to a technical field of a medical material. Disclosed are a light-emitting radical cation probe generated in situ by acid induction, and preparation therefor and application thereof. The light-emitting radical cation probe is obtained by oxidation of a compound of formula I in an acidic condition, and has a structure of formula II. A preparation method for the light-emitting radical cation probe is further disclosed. The radical cation probe of the present invention is generated by an in situ reaction in an acidic environment without a seperation, has the advantages of emitting red to near-infrared light, high generation efficiency and good stability, and can be used in fluorescence imaging. The radical cation probe of the present invention can achieve in situ imaging in an acidic stomach environment and gastrointestinal imaging. The probe of the present invention is further used in preparing a probe for monitoring and measuring a food digestion process in a stomach or preparing a reagent for monitoring and measuring treatment of anti-gastric drug that neutralizes gastric acid.

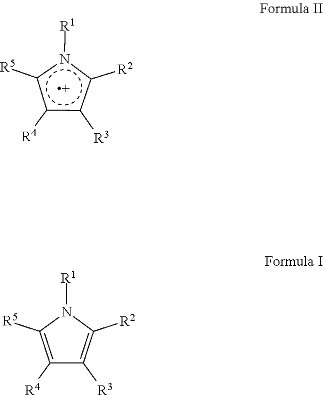

### TECHNICAL FIELD

The present invention belongs to a technical field of a medical material, and relates to a red to near-infrared light-emitting radical cation in situ generated in an acidic condition, and in particular, to a light-emitting pyrrole radical cation probe generated by acidic induction, preparation thereof, and application thereof in gastrointestinal imaging and detection for effect of anti-gastric drug.

### BACKGROUND

Gastrointestinal imaging with high spatial and temporal resolution and sensing technologies are crucial for the diagnosis of and medicine development for gastrointestinal diseases. For common clinical diseases, such as abnormal gastric emptying, it is necessary to analyze gastrointestinal structure and physiological function for accurate diagnosis. However, current clinical imaging technologies, such as ultrasound, computed tomography, and magnetic resonance imaging, are generally used only in assessing the gastrointestinal structure conditions; while gastrointestinal function analysis requires invasive and uncomfortable endoscopy or gastric juice extraction. To facilitate the diagnosis of and medicine development for gastrointestinal diseases, probes with non-invasive gastrointestinal imaging and in situ sensing capabilities are in desperate need.

With many notable advantages, such as great penetration capability and little autofluorescence interference etc., near-infrared fluorescence imaging is widely used in imaging-mediated surgery. In recent years, a series of red to near-infrared emitting organic fluorophores for gastrointestinal imaging and sensing have been developed, such as cyanine and benzodibenzodiazole derivatives, which have the advantages of high spatial and temporal resolution and easy operation. However, it is complicated to prepare these fluorophores with electron closed-shell structures, severely limiting their application. Compared with the traditional saturated electron fluorophores, the energy gap between the excited state and the ground state of a radical cation is much smaller, which makes it possible to develop small-sized near-infrared fluorophores. However, almost all radical cations reported in previous studies are non-luminescent, which severely limits their biological application.

Therefore, it is necessary to develop an easy-to-synthesize fluorescent probe of radical cation with in situ gastric imaging and gastrointestinal monitoring and measuring capabilities. The pyrrole derivative precursors of the present invention show application prospects of being in situ converted into pyrrole radical cations with red to near-infrared fluorescence in an acidic stomach environment, successfully achieving gastrointestinal imaging, and monitoring and measuring a digestion process in stomach and an effect of anti-gastric drug.

### SUMMARY

To overcome the deficiencies and drawbacks of the prior art, a primary objective of the present invention is to provide a light-emitting radical cation probe (i.e., a light-emitting radical cation probe in situ generated by acid induction) and a preparation method therefor. The red to near-infrared light-emitting radical cation probe of the present invention is obtained by in situ generation of a dimethylpyrrole derivative precursor in an acidic condition (e.g., in an acidic stomach). The probe of the present invention can achieve gastrointestinal fluorescence imaging with high spatial and temporal resolution.

Another objective of the present invention is to provide the application of the above-described light-emitting radical cation probe. The radical cation probe is used in preparing a gastrointestinal fluorescence imaging agent. The probe of the present invention is also used in monitoring and measuring a gastric emptying process. In addition, the radical cation probe of the present invention can also be used in monitoring and measuring an effect of anti-gastric drug.

The objectives of the present invention are achieved by the following technical solutions.

A light-emitting radical cation probe is obtained by oxidation of a compound of formula I in an acidic condition, and has a structure of formula II.

The structure of the probe is:

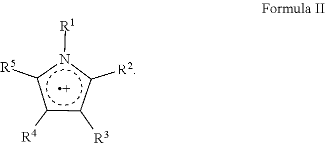

The compound of the formula I is:

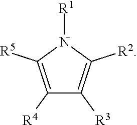

In the formula I and the formula II, R¹ is hydrogen, a substituted or unsubstituted aryl group, or a substituted or unsubstituted heteroaryl group.

Each of R² and R⁵ is independently a halogen, a substituted or unsubstituted alkyl group, an alkyloxy group, an alkylamino group, an aryl group, a heteroaryl group, an aryloxy group (Ar—O—), an arylamino group (Ar—NH—), an arylthio group (Ar—S—), a heteroaryloxy group, a heteroarylamino group, or a heteroarylthio group.

Each of R³ and R⁴ is independently hydrogen, the halogen, the substituted or unsubstituted alkyl group, the alkyloxy group, the alkylamino group, the aryl group, the heteroaryl group, the aryloxy group (Ar—O—), the arylamino group (Ar—NH—), the arylthio group (Ar—S—), the heteroaryloxy group, the heteroarylamino group, or the heteroarylthio group.

In R¹, the aryl group refers to a monocyclic or polycyclic aromatic group having 6-20 carbon atoms, and the representative aryl group includes a phenyl group, a naphthyl group, an anthryl group and a pyrenyl group.

The substituted aryl group means that a hydrogen on a ring of the aryl group is substituted by one or more of an alkoxy group, an amino group, and a carboxyl group.

The heteroaryl group refers to a monocyclic or polycyclic heteroaromatic group having 1-20 carbon atoms and 1-4 heteroatoms selected from N, S and O, and the representative heteroaryl group includes a pyrrolyl group, a pyridinyl group, a pyrimidinyl group, an imidazolyl group, a thiazolyl group, an indolyl group, an azanaphthyl group, an azaanthryl group and an azapyrenyl group.

In R², R³, R⁴ and R⁵, the alkyl group is a C₁₋₃₀ alkyl group, the alkyloxy group is a C₁₋₃₀ alkyloxy group, the alkylamino group is a C₁₋₃₀ alkylamino group, and the alkylthio group is a C₁₋₃₀ alkylthio group.

In R², R³, R⁴ and R⁵, the substituted alkyl group means that a hydrogen in the alkyl group is substituted by a hydroxyl group, a methoxyl group, the carboxyl group, or the halogen.

In R², R³, R⁴ and R⁵, the aryl group refers to the monocyclic or polycyclic aromatic group having 6-20 carbon atoms, and the representative aryl group includes the phenyl group, the naphthyl group, the anthryl group and the pyrenyl group. The aryl group in each of the aryloxy group, the arylamino group and the arylthio group is independently the monocyclic or polycyclic aromatic group having 6-20 carbon atoms, and the representative aryl group includes the phenyl group, the naphthyl group, the anthryl group and the pyrenyl group.

The heteroaryl group refers to the monocyclic or polycyclic heteroaromatic group having 1-20 carbon atoms and 1-4 heteroatoms selected from N, S and O, and the representative heteroaryl group includes the pyrrolyl group, the pyridinyl group, the pyrimidinyl group, the imidazolyl group, the thiazolyl group, the indolyl group, the azanaphthyl group, the azaanthryl group and the azapyrenyl group. The heteroaryl group in each of the heteroaryloxy group, the heteroarylamino group and the heteroarylthio group is the monocyclic or polycyclic heteroaromatic group having 1-20 carbon atoms and 1-4 heteroatoms selected from N, S and O, and the representative heteroaryl group includes the pyrrolyl group, the pyridinyl group, the pyrimidinyl group, the imidazolyl group, the thiazolyl group, the indolyl group, the azanaphthyl group, the azaanthryl group and the azapyrenyl group.

The light-emitting radical cation probe is stable in an acidic environment.

R¹ is the phenyl group or a methoxyphenyl group, each of R² and R⁵ is methyl group, R³ is —CH₂—OH, and R⁴ is hydrogen or piperidyl-1-methylene

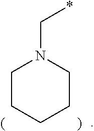

The acidic condition refers to pH≤3 (e.g., pH=1-3). The acidic condition may be an acidic solution or an acidic buffer solution.

The oxidation refers to oxidation with an oxidizing agent. The oxidizing agent is oxygen gas, a mono-electron oxidizing agent, or an oxygen-containing atmosphere.

A preparation method for the radical cation probe with near-infrared light emission (formula II) includes the following step: performing an oxidation reaction on the compound of the formula I in an acidic condition to obtain a near-infrared light-emitting radical cation probe (formula II).

The compound of formula II is:

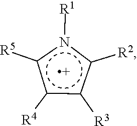

wherein R¹, R², R³, R⁴, and R⁵are as defined in the above formula I.

Reaction equation is as follows:

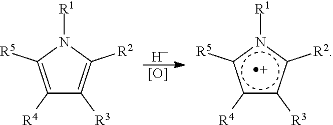

The reaction is performed in the acidic condition (pH≤3, e.g., pH=1-3).

The acidic condition refers to an acidic solution, referring to all acidic aqueous solutions at pH≤3 (pH=1-3), including the Britton-Robinson buffer and simulated gastric acid buffer (HCl).

The oxidation is performed under the action of an oxidizing agent. The oxidizing agent is oxygen gas and an atmosphere containing oxygen gas (e.g., air).

The compound of the formula I may be formulated into a solution in a solvent, which is then mixed with an acidic solution; and may also be directly mixed with the acidic solution. The concentration of the compound of the formula I in the solvent (with the solvent being dimethyl sulfoxide (DMSO), acetonitrile (MeCN), ethanol (CH₃CH₂OH), methanol (CH₃OH), and the like) is 1 μM to 1000 mM. The volume ratio of the acidic aqueous solution (pH=2-3) to the solution of the compound of the formula I is 99:1.

When the compound of the formula I is directly mixed with the acidic solution, the concentration of the compound of the formula I in the acidic solution is 1 μM to 1000 mM.

A time for the reaction is 5-120 min.

A temperature for the reaction is room temperature.

The radical cation probe is used in preparing a fluorescent probe for gastrointestinal imaging.

The near-infrared light-emitting probe based on the radical cation is used for in situ detecting a gastric emptying process (i.e., the probe of the present invention being used as a probe for in situ detecting the gastric emptying process). The gastric emptying process refers to a food digestion process in a gastrointestinal tract.

In addition, the radical cation probe of the present invention is used in monitoring and measuring an effect of anti-gastric drug.

The food refers to rice paste or water.

The anti-gastric drug refers to a medicine that neutralizes gastric acid in a stomach with excess gastric acid secreted.

The medicine that neutralizes gastric acid is preferably a sodium bicarbonate tablet.

The radical cation probe of the present invention is generated by an in situ reaction in an acidic environment. The radical cation probe does not need to be separated, has the advantages of emitting red to near-infrared light, high generation efficiency and good stability, and can be used in fluorescence imaging.

Compared with the prior art, the present invention has the following advantages and beneficial effects:
1. The fluorescent probe of the radical cation of the present invention can be in situ generated in the stomach and used in gastrointestinal fluorescence imaging with high spatial and temporal resolution.
2. The radical cation probe of the present invention has near-infrared fluorescence and can be used in monitoring and measuring the gastric emptying process and anti-gastric acid treatment.

### BRIEF DESCRIPTION OF DRAWINGS

FIG. 1 shows structural formulas of different pyrrole derivatives and pictures thereof in buffer solutions having different pH under daylight lamp: (A), the structural formulas of the pyrrole derivatives; and (B), the pictures of different pyrrole derivatives in buffer solutions having different pH under daylight lamp;

FIG. 2 shows an absorption spectrum and a fluorescence emission spectrum of a radical cation compound P3^{⋅+} converted from P3 (2 mM) by in situ oxidation in an acidic buffer solution (pH=2.0-2.6) under the action of air: (A), the absorption spectrum; and (B), the fluorescence emission spectrum with an excitation wavelength being 600 nm;

FIG. 3 shows an absorption spectrum and a fluorescence emission spectrum of a radical cation compound P6^{⋅+} converted from P6 (2 mM) by in situ oxidation in an acidic buffer solution (pH=2.0-3.0) under the action of air: (A), the absorption spectrum; (B), the fluorescence emission spectrum with an excitation wavelength being 600 nm; and (C), curves formed by absorption values and fluorescence intensity at different pH;

FIG. 4 shows an absorption spectrum and a fluorescence emission spectrum of a radical cation compound P7^{⋅+} converted from P7 (2 mM) by in situ oxidation in an acidic buffer solution (pH=2.0-3.0) under the action of air: (A), the absorption spectrum; (B), the fluorescence emission spectrum with an excitation wavelength being 600 nm; and (C), curves formed by absorption values and fluorescence intensity at different pH;

FIG. 5 shows an electron spin resonance (EPR) spectrum of P3^{⋅+} solid powder;

FIG. 6 shows ¹H NMR spectra of P3^{⋅+} in deuterated water (pH=2.0) and P3 in deuterated acetonitrile;

FIG. 7 shows an absorption spectrum, an emission spectrum, and a change of their intensities over time of P3^{⋅+} converted from P3 (5 mM) in an acidic buffer solution (pH=2.0): (A), the time-dependent absorption spectrum of P3^{⋅+}; (B), the time-dependent fluorescence emission spectrum of P3^{⋅+}; and (C) and (D), the relationships between the absorption intensity at 600 nm of P3^{⋅+} and time as well as between the fluorescence intensity at 680 nm of P3^{⋅+} and time, with an excitation wavelength being 600 nm;

FIG. 8 shows emission spectra and a change of intensity of the working solution of fluorescent dye Amplex Red with the change of P3 (P3^{⋅+}) concentrations: (A), the fluorescence emission spectra of Amplex Red with the change of P3 concentrations; and (B), the relationship between the fluorescence intensity at 590 nm of Amplex Red and the P3 concentration;

FIG. 9 shows absorption spectra, emission spectra, and a change of intensity of P3^{⋅+} converted from P3 in an acidic buffer solution (pH=2.0) containing different metal ions (Na⁺, K⁺, Mg²⁺, and Ca²⁺), anti-oxidizing agents (reduced glutathione and ascorbic acid), carbohydrates (glucose and soluble starch), and proteins (pepsin, hemoglobin, papain, ovalbumin, lactoferrin, lactoglobulin, transferrin, and bovine serum albumin): (A), the absorption spectra; (B), the fluorescence emission spectra with an excitation wavelength being 600 nm; and (C), the plots for change of ratio (I/I₀) of the fluorescence intensity at 680 nm of P3^{⋅+} in a pure acidic buffer solution (pH=2.0) containing different relevant interferents;

FIG. 10 shows fluorescence spectra and a change of intensity of P3^{⋅+} (2 mM) in glycerol/water mixture with increasing glycerol contents: (A), the fluorescence spectrum with an excitation wavelength being 600 nm; and (B), the diagram for change of ratio (I/I₀) of the emission intensity at 680 nm of P3^{⋅+} in glycerol/water mixture with increasing glycerol contents to the emission intensity at 680 nm of P3^{⋅+} in an aqueous solution;

FIG. 11 shows fluorescence lifetime spectra and change of fluorescence lifetime of P3^{⋅+} (2 mM) in glycerol/water mixture with increasing glycerol contents: (A), the fluorescence lifetime spectrum with an emission wavelength being 680 nm; and (B), the change values of fluorescence lifetime of P3^{⋅+} in glycerol/water mixture with increasing glycerol contents;

FIG. 12 shows in vivo fluorescence images and a change of gastric fluorescence intensity of nude mice over time, after a gavage of 70 μL of the aqueous solution containing 1.5 mg of P3: (A), the in vivo images of the mice over time after the gavage; and (B), the diagram for change of ratio (I/I₀) of the fluorescence intensity at different time to the fluorescence intensity at 0 min of the respective gastric region;

FIG. 13 shows in vivo fluorescence images over time of mice with an opened abdominal cavity and fluorescence images of dissected organs of lung, liver, spleen, kidney, and heart, after a gavage of 70 μL of the aqueous solution containing 1.5 mg of P3;

FIG. 14 shows fluorescence images of gastrointestinal anatomy of nude mice at various time points, after gavages of 150 μL of rise paste containing 1.5 mg of P3 and 70 μL of the aqueous solution containing 1.5 mg of P3 separately;

FIG. 15 is an electron spin resonance (EPR) spectrum of a blue content of a stomach collected by anatomy of nude mice, after a gavage of 150 μL of rise paste containing 1.5 mg of P3;

FIG. 16 shows a pre-experiment and a modeling experiment for monitoring and measuring anti-gastric acid treatment of a mice: (A), in vivo images of two groups of normal nude mice after gavages of 200 μL of the acidic buffer solution (pH 2.0) and the neutral buffer solution (pH 7.0) containing 1.5 mg of P3; and (B), in vivo fluorescence images of normal nude mice and anti-gastric acid model mice after a gavage of 70 μL of the aqueous solution containing 1.5 mg of P3; and

FIG. 17 shows microscopic images and analysis for hepatorenal functions of tissue sections of nude mice after a gavage of 70 μL of the aqueous solution containing 1.5 mg of P3 and a normal nude mice: (A), the micrographs of the tissue sections; and (B), the hepatorenal function indexes.

### DETAILED DESCRIPTION OF EMBODIMENTS

The present invention will be further described in detail below with reference to Examples for the purpose of better understanding the research contents of the present invention, but embodiments of the present invention are not limited thereto.

### EXAMPLE 1: IN SITU GENERATION OF COMPOUND P3^{⋅+} IN AN ACIDIC BUFFER SOLUTION (PH=2.0-2.6)

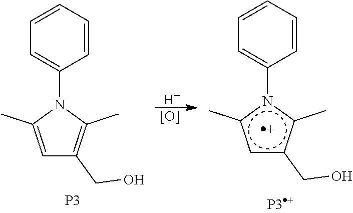

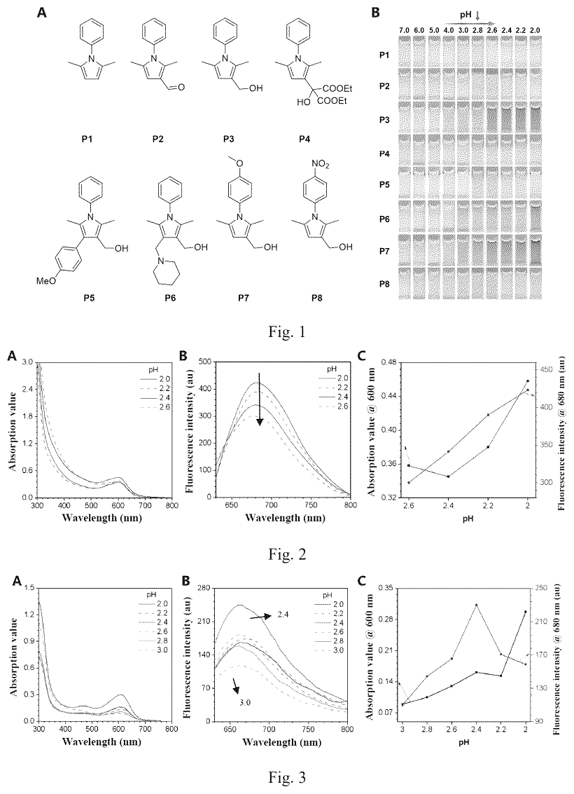

30 μL of DMSO stock solution (200 mM) of the compound P3 (2,5-dimethyl-3-hydroxymethylene-1-phenyl-¹H-pyrrole) was added to 2970 μL of the acidic buffer solution (pH=2.0-2.6) (P3 may be directly added to the acidic buffer solution and then sonicated for 5 min, and with the acidic buffer solution herein specifically referring to a buffer solution at pH=2.0-2.6 formulated by glacial acetic acid, boric acid, phosphoric acid, and sodium hydroxide). After being mixed well, the protonated pyrrole compound P3 was rapidly converted into the respective radical cation compound P3^{⋅+} (with the conversion being performed in an atmosphere of air). The generation of P3^{⋅+} may be proved by bright field pictures and tests of an ultraviolet-visible absorption spectrum and a fluorescence spectrum. The results were shown in FIG. 1 and FIG. 2.

### EXAMPLE 2: IN SITU GENERATION OF P6^{⋅+} IN AN ACIDIC BUFFER SOLUTION (PH=2.0-3.0)

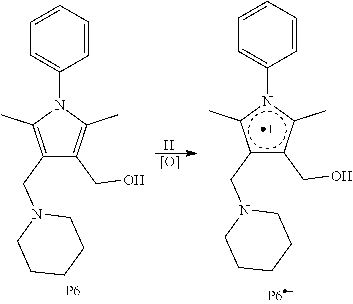

30 μL of DMSO stock solution (200 mM) of the compound P6 (4-(piperidine-1-methylene)-2,5-dimethyl-3-hydroxymethylene-¹H-pyrrole) was added to 2970 μL of the acidic buffer solution (pH=2.0-3.0). After being mixing well, the pyrrole compound P6 would be converted into the respective radical cation compound P6^{⋅+} within 30 min (with the conversion being performed in an atmosphere of air). The generation of P6^{⋅+} may be proved by bright field pictures and tests of an ultraviolet-visible absorption spectrum and a fluorescence spectrum. The results were shown in FIG. 1 and FIG. 3.

### EXAMPLE 3: IN SITU GENERATION OF P7^{⋅+} IN AN ACIDIC BUFFER SOLUTION (PH=2.0-3.0)

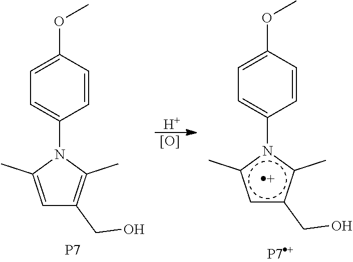

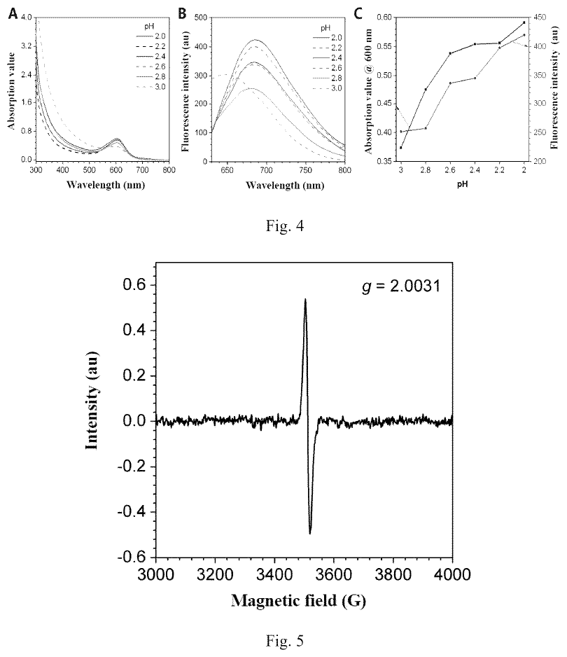

30 μL of DMSO stock solution (200 mM) of the compound P7 (1-(4-methoxyphenyl)-2,5-dimethyl-3-hydroxymethylene-¹H-pyrrole) was added to 2970 μL of the acidic buffer solution (pH=2.0-3.0). After being mixing well, the protonated pyrrole compound P7 would be converted into the respective radical cation compound P7^{⋅+} within 30 min (with the conversion being performed in an atmosphere of air). The generation of P7^{⋅+} may be proved by bright field pictures and tests of an ultraviolet-visible absorption spectrum and a fluorescence spectrum. The results were shown in FIG. 1 and FIG. 4.

### EXAMPLE 4

### Verifying Generation of Pyrrole Radical Cation

### (1) Electron Paramagnetic Resonance (EPR) Test

10 mg of the compound P3 was added to 1 mL of the Britton-Robinson (BR) buffer solution (pH 2.0) and then sonicated (480 W, 10 min) to obtain a blue solution, and the blue solution was freeze-dried to obtain blue powder. The blue solid powder was then placed in a EPR tube, and a EPR spectrum was recorded on a Bruker E500-10/12 electron paramagnetic resonance CW (20 mW) within 5 min. As shown in FIG. 5, an obvious signal peak of a radical with g value being 2.0031 was collected, which proved the generation of the pyrrole radical cation P3^{⋅+}.

### (5) Nuclear Magnetic Resonance Characterization

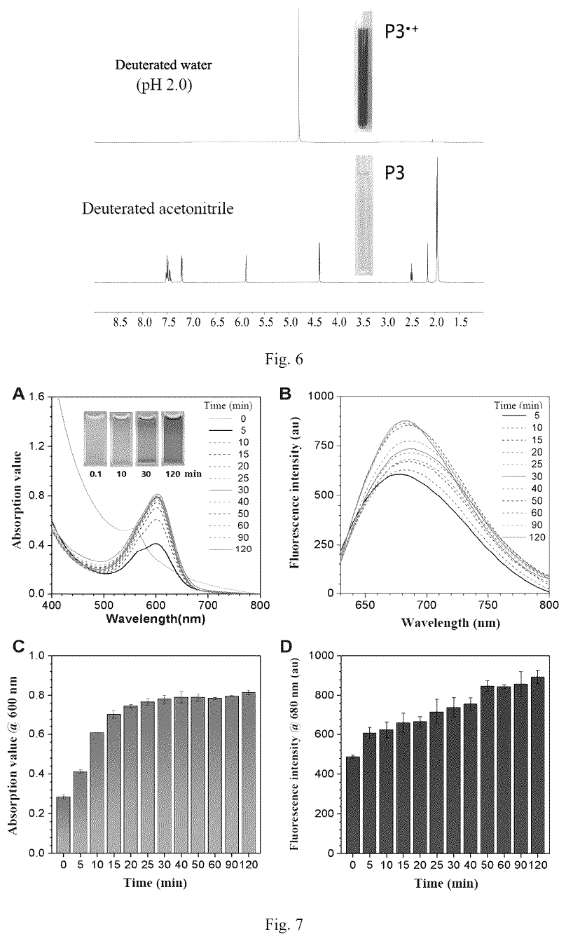

5.5 mg of the compound P3 was separately added to 550 μL of deuterated water (pH 2.0) and 550 μL of deuterated acetonitrile and then sonicated (480 W, 10 min). The ¹H NMR of the deuterated solutions was then recorded on the Bruker AV 400 NMR spectromete, and the spectrums were finally processed for comparison. It can be seen from FIG. 6 that there was no nuclear magnetic resonance (NMR) signal of the compound P3 in the acidic deuterated water, which laterally illustrated that the radical cation P3^{⋅+} was generated from P3 in the deuterated water (pH 2.0).

### EXAMPLE 5

Formation Process and Mechanism Verification of P3^{⋅+}

### (1) Absorption Spectrum, Fluorescence Emission Spectrum, and Respective Intensity Change over Time of P3^{⋅+}

30 μL of DMSO stock solution (200 mM) of the compound P3 was added to 2970 μL of the acidic buffer solution (with the acidic buffer solution herein specifically referring to a buffer solution with pH of 2.0 formulated by glacial acetic acid, boric acid, phosphoric acid, and sodium hydroxide). After being mixed well, tests of the time-dependent ultraviolet-visible absorption spectrum and fluorescence spectrum were performed (2 h). As shown in FIG. 7, a stable absorption peak at 600 nm and a fluorescence emission peak at 680 nm can be observed for the compound P3 in the acidic buffer solution (pH 2.0). It can be seen from the absorption peak that P3 can be converted into red to near-infrared light-emitting radical cation P3^{⋅+} within 5 min (with 570 nm being converted into 600 nm). The illustration in FIG. 7A refers to a picture of the compound P3 under daylight at different reaction time points (0.1, 10, 30, and 120 min), after being added to the acidic buffer solution (pH 2.0).

### (2) Verifying Trace Amount of H₂O₂ Produced during Generation of P3^{⋅+}

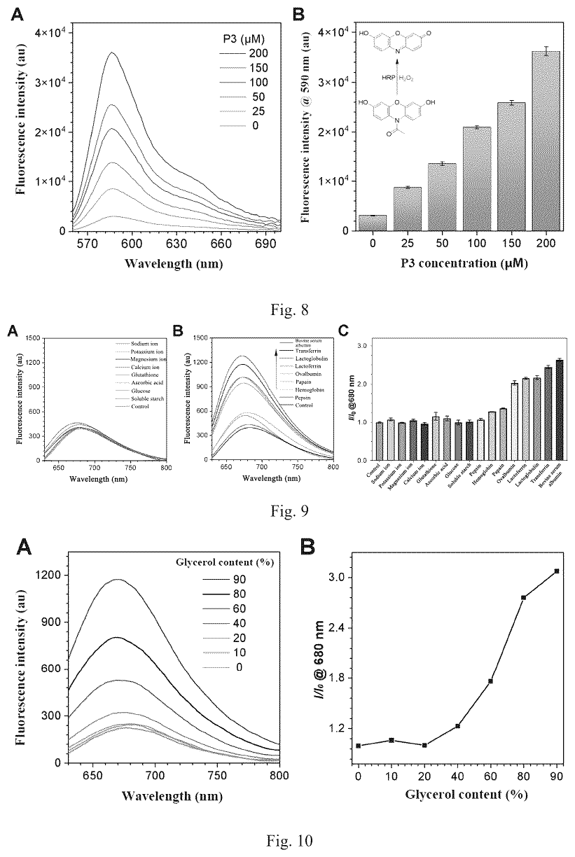

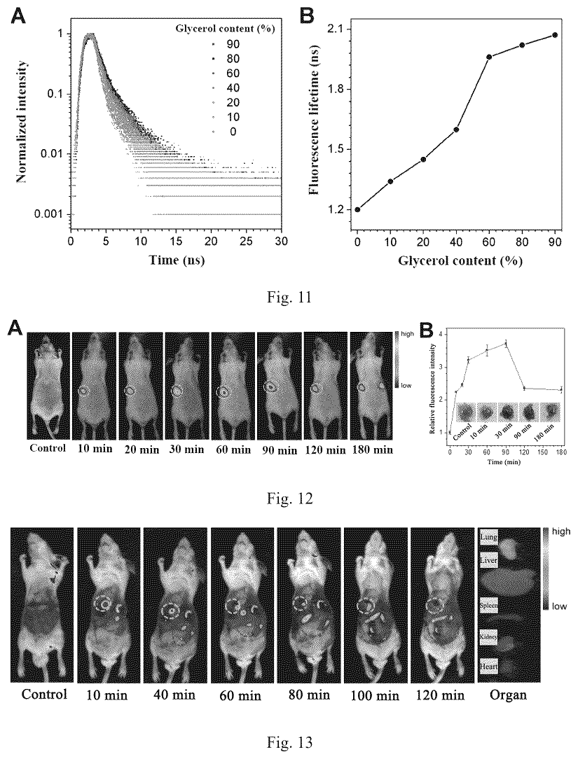

2 μL of the blue solutions with different concentrations of P3^{⋅+} (P3: 0, 25, 50, 100, 150, and 200 μM) were added to 48 μL of the PBS solutions, and then 50 μL of the Amplex Red working solution (being formulated by mixing 4.85 mL of PBS, 100 μL of 10 U/mL HRP enzyme, and 50 μL of DMSO stock solution of 10 mM Amplex Red (Maclean)) was added to the PBS solutions. Three replicate holes were made for each concentration. After the incubation at 30° C. for 30 min, the fluorescence emission spectrum test was performed, with an excitation wavelength being 530 nm and an emission wavelength being 560-700 nm. As can be seen from FIG. 8, the fluorescence of the Amplex Red working solution (H₂O₂ probe) increased at 590 nm as the concentration of P3^{⋅+} (P3) increased, which indicated that the generation process of P3^{⋅+} will produce a trace amount of H₂O₂.

### EXAMPLE 6

### Anti-Interference Experiment of P^{⋅+}

An acidic buffer solution (with the acidic buffer solution herein specifically referring to a buffer solution with pH of 2.0 formulated by glacial acetic acid, boric acid, phosphoric acid, and sodium hydroxide) (pH 2.0) containing different metal ions (Na⁺, K⁺, Mg²⁺, and Ca²⁺), anti-oxidizing agents (reduced glutathione and ascorbic acid), carbohydrates (glucose and soluble starch), and proteins (pepsin, hemoglobin, papain, ovalbumin, lactoferrin, lactoglobulin, transferrin, and bovine serum albumin) was pre-formulated. 30 μL of DMSO stock solution (200 mM) of the compound P3 was added to 2970 μL of the above-described acidic buffer solution, incubated for 2 h after being mixed well, and converted into a blue P3^{⋅+} solution, on which the tests of ultraviolet-visible absorption spectrum and fluorescence spectrum were performed with an excitation wavelength being 600 nm. The fluorescence image of the respective sample was obtained by a small animal imaging instrument. It can be seen from FIG. 9 that different metal ions, common anti-oxidizing agents, and carbohydrates would not affect the fluorescence of P3^{⋅+}, and some proteins also significantly increased the fluorescence of P3^{⋅+}.

### EXAMPLE 7

### Verifying Increasing Fluorescence of Proteins (Viscosity Experiment)

Glycerol and water were mixed based on different volume ratios (water/glycerol=100/0, 90/10, 80/20, 60/40, 40/60, 20/80, and 10/90) to form mixed solutions with different glycerol contents. The pre-formulated P3^{⋅+} in the acidic buffer solution (pH 2.0) was dissolved into these mixed solutions with the final concentration of the compound P3 being 2 mM. Then, the tests of fluorescence emission spectrum (with the excitation wavelength being 600 nm) and fluorescence lifetime curve (with emission wavelength being 680 nm) were performed. The fluorescence lifetime was obtained by origin double exponential fitting. As shown in FIGS. 11-12, with the continuous increase of the glycerol content (increase from 0% to 90%), the ratio of relative fluorescence intensity (I/I₀, with I and I₀ referring to the fluorescence intensity of P3^{⋅+} in different glycerol contents and the fluorescence intensity of P3^{⋅+} in the aqueous solution, respectively) and fluorescence lifetime (fitting value) of the radical cation P3^{⋅+} both gradually increased, which indicated that the fluorescence intensity and photophysical performance of the compound P3 will be further enhanced in the case of limited molecular motion.

### EXAMPLE 8

Animal experiments: male eight-week-old Balb/c nude mice were purchased from Guangzhou Yancheng Biotechnology Co. Ltd. The mice were adaptively housed for one week and fasted for 24 hours before gavage.

### (1) In Vivo Imaging

The mice were anesthetized beforehand, 1.5 mg of the compound P3 (DMSO) was dispersed in 70 μL of ultrapure water, and then the molecule was administered into the stomach of the mice by gavage. Subsequently, the in vivo fluorescence imaging was performed at room temperature by using the small animal imaging instrument Bruker MI SE 721. The imaging was performed every 10 min until 60 min, every 30 min until 120 min, and then every 60 min until 180 min. Two imaging steps were performed sequentially: 590 nm/700 nm, with exposure time being 60 s, and white light exposure time being 0.175 s. As shown in FIG. 12, an obvious near-infrared fluorescence signal was observed in the stomach of the mice at 10 min; an obvious near-infrared fluorescence signal is observed in the intestinal region at 180 min. In the same conditions, quantitative analysis of fluorescence imaging was performed by using the instrument Bruker MI SE 721. The quantitative analysis of fluorescence was performed on the gastric signals in the mice at all the time points above. The mean value of fluorescence at three sites in the gastric region at 0 min was taken as I₀, and the mean value of fluorescence at three sites in the gastric region at other time points was taken as I. A semi-quantitative analysis of the change of the gastric signals in the mice was performed by means of the fluorescence ratio (I/I₀). In addition, the mice were euthanized at different time points, and the gastrointestinal tracts of the mice were dissected out for white light photography. Then, the blue content of stomach could be observed.

### (2) In Vivo Imaging with Laparotomy

The mice were anesthetized beforehand, and the gastrointestinal tract was then exposed by a single incision in the abdomen of the mice. 1.5 mg of the compound P3 (DMSO) was dispersed in 70 μL of ultrapure water, and then the molecule was administered into the stomach of the anesthetized mice by gavage. Subsequently, the in vivo fluorescence imaging was performed at 37° C. by using the small animal imaging instrument Bruker MI SE 721. The imaging was performed every 20 min until 120 min. Two imaging steps were performed sequentially: 590 nm/700 nm, with exposure time being 60 s, and white light exposure time being 0.175 s. As shown in FIG. 13, the fluorescence signal in the stomach of the mice is strongest at 20 min, while the intestinal fluorescence signal increases with time. Finally, the mice were euthanized and other visceral organs than the gastrointestinal tract were dissected out without any fluorescence signal.

### (3) Monitoring and Measuring of Gastric Digestion Process

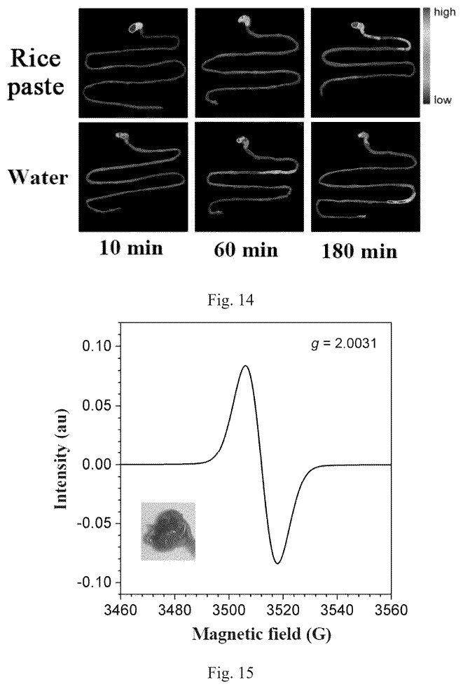

The mice were anesthetized beforehand, and then the P3/rise paste mixture (with 1.5 mg of P3 (DMSO) being dispersed in 150 μL of commercially brewed rise paste) and the P3 aqueous solution (with 1.5 mg of P3 (DMSO) being dispersed in 70 μL of ultrapure water) were separately administrated into the stomach of the mice. The mice were euthanized for anatomy at different time points (10 min, 60 min, and 180 min), and ex vivo fluorescence imaging of the dissected gastrointestinal tract was performed. Two imaging steps were performed sequentially: 590 nm/700 nm, with exposure time being 60 s, and white light exposure time being 0.175 s. In FIG. 14, by comparing the rise paste group containing the compound P3 with the water group containing the compound P3, it can be seen that for the rise paste group, there was no obvious attenuation of gastric fluorescence signal within 180 min, which indicated that it took longer time (>180 min) for the emptying of food; while for the water group, the stomach was emptied at 180 min with only a weak fluorescence signal in the intestinal tract, which indicated that the digestion rate of pure water in the gastrointestinal tract is faster.

### (4) Verifying In Situ Generation of P3^{⋅+} in Stomach

The P3/rise paste mixture (as above) was administrated into the stomach of anesthetized mice, and the mice were incubated at room temperature for 2 hours. The mice were euthanized, and the stomach was dissected out. The blue gastric contents of each parallel group of mice were collected and dehydrated with a freeze dryer to obtain a blue solid. The blue solid powder was then placed in an EPR tube, and an EPR spectrum was recorded on a Bruker E500-10/12 electron paramagnetic resonance CW (20 mW) within 5 min. In FIG. 15, a signal peak with g value being 2.0031 in the EPR spectrum indicated the generation of the pyrrole radical cation in the stomach.

### (5) Monitoring and Measuring of Anti-Gastric Acid Treatment

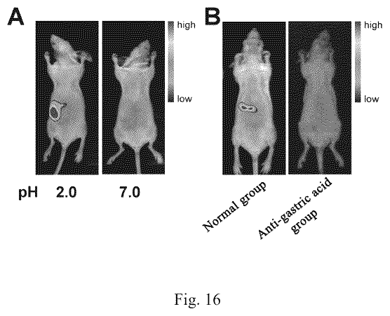

Before modeling, a pre-experiment was performed: 1.5 mg of P3 (DMSO) was dispersed in the buffer solutions (200 μL, with pH of 2.0 or 7.0). Then, the two groups of solutions were separately administrated into the stomach of two groups of mice, and the mice were incubated for 2 hours for in vivo fluorescence imaging. Two imaging steps were performed sequentially: 590 nm/700 nm, with exposure time being 60 s, and white light exposure time being 0.175 s. As can be seen from FIG. 16A, only for the acidic group administrated into the stomach of the mice, a fluorescence signal can be generated. Besides, for the neutral group, there was not any signal.

The mice were randomly divided into a normal group and an anti-acid group. The mice in the anti-acid group were induced with a sodium bicarbonate tablet (250 mg kg⁻¹, oral), and the mice in the normal group were given 0.2 mL of normal saline 1 h before administration. Then, the P3 aqueous solution (with 1.5 mg of P3 (DMSO) dispersed in 70 μL of ultrapure water) was separately administrated into the stomach of the two groups of mice, and the mice were incubated for 2 hours for in vivo fluorescence imaging. Two imaging steps were performed sequentially: 590 nm/700 nm, with exposure time being 60 s, and white light exposure time being 0.175 s. As can be seen from FIG. 16B, there was not any fluorescence signal for the anti-gastric acid treatment group, compared with the strong fluorescence signal of the normal group.

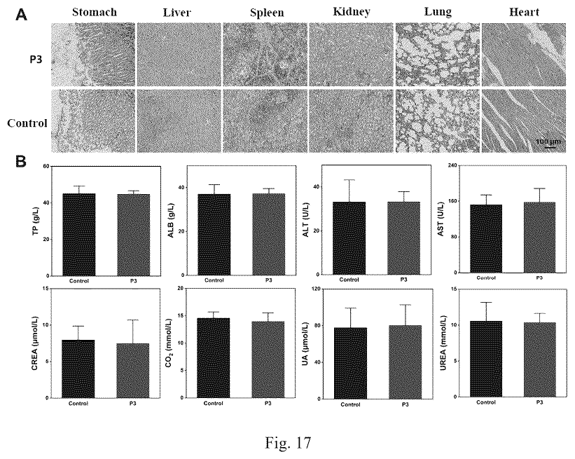

### (6) H&E Staining and Hepatorenal Index Analysis (FIG. 17)

The mice were randomly selected for gavage of the P3 solution (with 1.5 mg of P3 dispersed in 70 μL of water). 2 hours after gavage, blood (approximately 500 μL) was collected through the ocular fundus venous plexus and stored in an icebox 20 minutes before centrifugation at 3,500 r.p.m. to prevent clotting. Roche Cobas E602 electrochemical light-emitting immunoassay analyzer was used to test and analyze total protein (TP), albumin (ALB), alanine aminotransferase (ALT), aspartate aminotransferase (AST), creatinine (CREA), carbon dioxide (CO₂), uric acid (UA), urea (UREA) in serum samples (200 μL). The stomach, liver, spleen, kidney, and heart of the mice were taken for histological examination in 4% paraformaldehyde (PFA) 2 hours after gavage of the P3 solution.

All tissues were fixed with 4% PFA, dehydrated with an ethanol solution, embedded with paraffin, and cut into 20 mm coronal and low-temperature sections for hematoxylin and eosin (H&E) staining. Paraffin was removed by xylene washing. Then, the sections were incubated with hematoxylin and eosin for 4 min and washed with distilled water. Stained sections were examined by using the Nikon ECLIPSE 80i microscope.

As shown in FIG. 17A, compared with the control group, the major visceral organs (stomach, liver, spleen, kidney, lung, and heart) of the mice treated with P3 showed no obvious injury or inflammatory lesion. In addition, as shown in FIG. 17B, the expression levels of the biochemical indexes of the hepatorenal function in the P3-treated mice were also similar to those in the control group.

In conclusion, these results verify that the pyrrole radical cation P3^{⋅+} in situ generated in the stomach has good biocompatibility and is suitable for in vivo gastrointestinal imaging.

The P6^{⋅+} and the P7^{⋅+} of the present invention can also perform the gastrointestinal imaging, however, their imaging effect is not as good as that of P3^{⋅+}, due to the lower conversion efficiency and the weaker light emission of the P6 molecule.

The above Examples are preferred embodiments of the present invention, but the embodiments of the present invention are not limited by the above Examples. Any other changes, modifications, substitutions, combinations, or simplifications that do not go beyond the spirit and principle of the present invention should all be equivalent substitution modes and are included in the protection scope of the present invention.

1. A light-emitting radical cation probe, characterized in that, the probe is obtained by oxidation of a compound of formula I in an acidic condition, and has a structure of formula II;
the structure of the probe is: 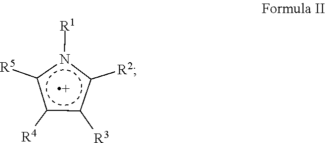
the compound of the formula I is: 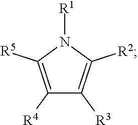
in the formula I and the formula II, R¹ is hydrogen, a substituted or unsubstituted aryl group, or a substituted or unsubstituted heteroaryl group;
each of R² and R⁵ is independently a halogen, a substituted or unsubstituted alkyl group, an alkyloxy group, an alkylamino group, an aryl group, a heteroaryl group, an aryloxy group, an arylamino group, an arylthio group, a heteroaryloxy group, a heteroarylamino group, or a heteroarylthio group; and
each of R³ and R⁴ is independently hydrogen, the halogen, the substituted or unsubstituted alkyl group, the alkyloxy group, the alkylamino group, the aryl group, the heteroaryl group, the aryloxy group, the arylamino group, the arylthio group, the heteroaryloxy group, the heteroarylamino group, or the heteroarylthio group.

2. The light-emitting radical cation probe according to claim 1, characterized in that,
in R¹, the aryl group is a phenyl group, a naphthyl group, an anthryl group or a pyrenyl group, and the substituted aryl group means that hydrogen on a ring of the aryl group is substituted by one or more of an alkoxy group, an amino group and a carboxyl group;
the heteroaryl group is a pyrrolyl group, a pyridinyl group, a pyrimidinyl group, an imidazolyl group, a thiazolyl group, an indolyl group, an azanaphthyl group, an azaanthryl group, or an azapyrenyl group;
in R², R³, R⁴ and R⁵, the alkyl group is a C₁₋₃₀ alkyl group, the alkyloxy group is a C₁₋₃₀ alkyloxy group, the alkylamino group is a C₁₋₃₀ alkylamino group, and the alkylthio group is a C₁₋₃₀ alkylthio group;
in R², R³, R⁴ and R⁵, the substituted alkyl group means that hydrogen in the alkyl group is substituted by a hydroxyl group, a methoxyl group, the carboxyl group, or the halogen;
in R², R³, R⁴ and R⁵, the aryl group is the phenyl group, the naphthyl group, the anthryl group or the pyrenyl group, and the aryl group in each of the aryloxy group, the arylamino group and the arylthio group is independently the phenyl group, the naphthyl group, the anthryl group or the pyrenyl group;
the heteroaryl group is the pyrrolyl group, the pyridinyl group, the pyrimidinyl group, the imidazolyl group, the thiazolyl group, the indolyl group, the azanaphthyl group, the azaanthryl group or the azapyrenyl group, and heteroaryl group in each of the heteroaryloxy group, the heteroarylamino group and the heteroarylthio group is the pyrrolyl group, the pyridinyl group, the pyrimidinyl group, the imidazolyl group, the thiazolyl group, the indolyl group, the azanaphthyl group, the azaanthryl group or the azapyrenyl group;
the light-emitting radical cation probe is stable in an acidic environment; and
the acidic condition refers to pH≤3.

3. The light-emitting radical cation probe according to claim 2, characterized in that,
R¹ is the phenyl group or a methoxyphenyl group, each of R² and R⁵ is methyl group, R³ is —CH₂—OH, and R⁴ is hydrogen or piperidyl-1-methylene.

4. A preparation method for the light-emitting radical cation probe according to any one of claims 1 to 3, characterized in that, it comprises the following step: performing an oxidation reaction on the compound of the formula I in an acidic condition at pH≤3, to obtain a radical cation probe emitting red to near-infrared light,
the compound of the formula I is: 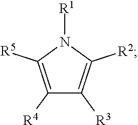
the light-emitting radical cation probe has a structure of the formula II: 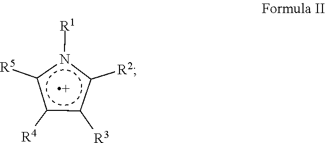
in the formula I and the formula II, R¹ is hydrogen, the substituted or unsubstituted aryl group, or the substituted or unsubstituted heteroaryl group;
each of R² and R⁵ is independently the halogen, the substituted or unsubstituted alkyl group, the alkyloxy group, the alkylamino group, the aryl group, the heteroaryl group, the aryloxy group, the arylamino group, the arylthio group, the heteroaryloxy group, the heteroarylamino group, or the heteroarylthio group; and
each of R³ and R⁴ is independently hydrogen, the halogen, the substituted or unsubstituted alkyl group, the alkyloxy group, the alkylamino group, the aryl group, the heteroaryl group, the aryloxy group, the arylamino group, the arylthio group, the heteroaryloxy group, the heteroarylamino group, or the heteroarylthio group.

5. The preparation method according to claim 4, characterized in that,
the reaction is performed in an aerobic condition; the acidic condition refers to an acidic solution with pH≤3; and
a concentration of the compound of the formula I in the acidic solution is 1 μM to 1000 mM.

6. The preparation method according to claim 4, characterized in that,
being aerobic refers to oxygen gas or an atmosphere containing oxygen gas; and a time for the reaction is 5-120 min.

7. An application of the light-emitting radical cation probe according to claim 1, characterized in that, the fluorescence-emitting radical cation probe is used in preparing a fluorescent probe or an imaging agent for in situ imaging in an acidic stomach environment and imaging with high spatiotemporal resolution for a gastrointestinal tract.

8. The application of the light-emitting radical cation probe according to claim 1, characterized in that, the fluorescence-emitting radical cation probe is used in preparing a probe for monitoring and measuring a food digestion process in a stomach; or the fluorescence-emitting radical cation probe is used in preparing a reagent for monitoring and measuring anti-gastric acid treatment.

9. The application of the light-emitting radical cation probe according to claim 1, characterized in that, the fluorescence-emitting radical cation probe is used in screening an anti-gastric acid medicine.

10. The application of the light-emitting radical cation probe according to claim 1, characterized in that, the light-emitting radical cation probe is used in a probe for detection of hydrogen ions.
